(19) United States
(12) Patent Application Publication
(10) Pub. No.: US 2017/0073293 A1
(43) Pub. Date: Mar. 16, 2017

(54) OXIDATION OF SOLIDS BIO-CHAR FROM LEVULINIC ACID PROCESSES

(71) Applicant: GFBiochemicals Limited, Valletta (MT)

(72) Inventors: Brian D. Mullen, Delano, MN (US); Erich J. Molitor, Elk River, MN (US); Alan K. Schrock, Pensacola, FL (US)

(21) Appl. No.: 15/123,511

(22) PCT Filed: Mar. 2, 2015
(86) PCT No.: PCT/US2015/018230

§ 371 Date: Sep. 2, 2016

Related U.S. Application Data
(60) Provisional application No. 61/947,154, filed on Mar. 3, 2014.

(51) Int. Cl. C07C 51/31; B01J 23/72

(52) U.S. Cl. CPC C07C 51/31; C07C 51/316; B01J 23/72

(57) ABSTRACT

The invention describes processes to convert biomass char, such as levulinic acid process char, into useful products.

### CROSS REFERENCE TO RELATED APPLICATIONS

This application claims priority to U.S. Provisional Patent Application No. 61/947,154, filed Mar. 3, 2014, entitled “Oxidation of Solids Bio-Char from Levulinic Acid Processes”, the contents of which are incorporated herein in their entirety for all purposes.

### FIELD OF THE INVENTION

The invention relates generally to conversion of acid-catalyzed carbohydrate decomposition products, such as char, into small molecules.

### BACKGROUND OF THE INVENTION

Levulinic acid (LA) can be used to make resins, plasticizers, specialty chemicals, herbicides and as a flavor substance. Levulinic acid is useful as a solvent, and as a starting material in the preparation of a variety of industrial and pharmaceutical compounds such as diphenolic acid (useful as a component of protective and decorative finishes), calcium levulinate (a form of calcium for intravenous injection used for calcium replenishment and for treating hypocalcemia. The use of the sodium salt of levulinic acid as a replacement for ethylene glycols as an antifreeze has also been proposed.

Esters of levulinic acid are known to be useful as plasticizers and solvents, and have been suggested as fuel additives. Acid catalyzed dehydration of levulinic acid yields alpha-angelica lactone.

Levulinic acid has been synthesized by a variety of chemical methods. But levulinic acid has not attained much commercial significance due in part to the high cost of the raw materials needed for synthesis. Another reason is the low yields of levulinic acid obtained from most synthetic methods. Yet, another reason is the formation of a formic acid byproduct during synthesis and its separation from the levulinic acid. Therefore, the production of levulinic acid has had high associated equipment costs. Despite the inherent problems in the production of levulinic acid, however, the reactive nature of levulinic acid makes it an ideal intermediate leading to the production of numerous useful derivatives.

Cellulose-based biomass, which is an inexpensive feedstock, can be used as a raw material for making levulinic acid. The supply of sugars from cellulose-containing plant biomass is immense and replenishable. Most plants contain cellulose in their cell walls. For example, cotton comprises 90% cellulose. Furthermore, it has been estimated that roughly 75% of the approximate 24 million tons of biomass generated on cultivated lands and grasslands are waste. The cellulose derived from plant biomass can be a suitable source of sugars to be used in the process of obtaining levulinic acid. Thus, the conversion of such waste material into a useful chemical, such as levulinic acid, is desirable.

Conversion of the biomass and carbohydrates into levulinic acid is often accompanied by char as a byproduct. Ideally, a decrease in the amount of char produced by the process is desirable. For example, char can foul the reaction container and can lead to a decrease in yield of desired products such as levulinic acid and formic acid. Char is a byproduct of the synthetic routes to produce levulinic that has not received a great deal of attention, other than to discard the char. As such, production of char results in a diminishment of potential valuable products from the biomass as well as a need to dispose of the char.

Therefore a need exists to address the disadvantages associated with the formation of char in conversions of biomass into useful products.

### BRIEF SUMMARY OF THE INVENTION

A major issue in producing levulinic acid is the separation of levulinic acid from the byproducts, especially from formic acid and char. Current processes generally require high temperature reaction conditions, generally long digestion periods of biomass, specialized equipment to withstand hydrolysis conditions, and as a result, the yield of the levulinic acid is quite low, generally in yields of 30 weight percent or less with formation of char and formic acid.

Therefore, a need exists for a new approach that overcomes one or more of the current disadvantages noted above.

The present embodiments surprisingly provide novel approaches for the conversion of biomass based char into water soluble products. The methods include subjecting biomass based char to an oxidant (and optionally a catalyst) to provide a mixture, whereby the biomass based char of the mixture is converted into water soluble products. Generally, the biomass based char is a byproduct from a biomass based material treated with a strong mineral acid, such as sulfuric, hydrochloric or methane sulfonic acid in an aqueous medium. The strong mineral acid may also be a heterogeneous acid catalyst, such as a strong cation exchange resin catalyst. The acid catalyst may also be derived from a Lewis acid catalyst.

Suitable biomass or carbohydrate based materials, include for example, furfuryl alcohol, C5 sugars, C6 sugars, lignocelluloses, cellulose, starch, polysaccharides, disaccharides, monosaccharides or mixtures thereof.

In one particular aspect, the carbohydrate is glucose, fructose, sucrose or combinations thereof.

Suitable metal containing catalysts contain for example, platinum, palladium, ruthenium, copper, cobalt, vanadium, tungsten, iron, silver, manganese, or gold. Specific catalysts may include, but are not limited to MeReO₃ (methyltrioxorhenium (VII)), RuCl₃, polyoxometalates, such as [AlMn^{II/III}(OH₂)W₁₁O₃₉]^{6-/7} copper compounds, such as copper sulfate or copper oxide, cobalt compounds, platinum catalysts such at supported platinum or complexes, Perovskite-type complexes (LaMnO₃), metal bromide catalysts, such as Co—Mn—Br, or Au/TiO₂, Suitable oxidants include, for example, oxygen, permanganates, nitric oxide, oxone, sodium nitrite, hypochlorite, ozone, or a peroxide, such as hydrogen peroxide.

The reaction between the biomass based char and the oxidant is generally conducted in an aqueous environment between a temperature of from about 20° C. to about 200° C.

In another aspect, char remains in the reaction vessel after the biomass based char is treated with an oxidant for a sufficient period of time, thereby providing water soluble products that can later be isolated for useful applications. It has been found that these water soluble products include, for example, one or more of levulinic acid, acetic acid, succinic acid, formic acid or mixtures thereof.

In another aspect, the char has been completely converted into water soluble products.

While multiple embodiments are disclosed, still other embodiments of the present invention will become apparent to those skilled in the art from the following detailed description. As will be apparent, the invention is capable of modifications in various obvious aspects, all without departing from the spirit and scope of the present invention. Accordingly, the detailed descriptions are to be regarded as illustrative in nature and not restrictive.

### BRIEF DESCRIPTION OF THE DRAWINGS

FIG. 1 is an ¹H NMR spectrum of water-soluble products from the oxidation of char described in Example 6.

FIG. 2 is a LC-MS chromatogram (top) scan of components from Example 6 with molecular ion of 117 M⁻ (negative mode) that shows a peak for succinic acid. LC-MS chromatogram (middle) scan of components from Example 6 with molecular ion of 115 M⁻ (negative mode) that shows a peak for levulinic acid. LC-MS chromatogram (bottom) displaying total ion count from Example 6 (negative mode) that shows a peaks for levulinic acid and succinic acid.

### DETAILED DESCRIPTION

In the specification and in the claims, the terms “including” and “comprising” are open-ended terms and should be interpreted to mean “including, but not limited to . . . ” These terms encompass the more restrictive terms “consisting essentially of” and “consisting of.”

It must be noted that as used herein and in the appended claims, the singular forms “a”, “an”, and “the” include plural reference unless the context clearly dictates otherwise. As well, the terms “a” (or “an”), “one or more” and “at least one” can be used interchangeably herein. It is also to be noted that the terms “comprising”, “including”, “characterized by” and “having” can be used interchangeably.

Unless defined otherwise, all technical and scientific terms used herein have the same meanings as commonly understood by one of ordinary skill in the art to which this invention belongs. All publications and patents specifically mentioned herein are incorporated by reference in their entirety for all purposes including describing and disclosing the chemicals, instruments, statistical analyses and methodologies which are reported in the publications which might be used in connection with the invention. All references cited in this specification are to be taken as indicative of the level of skill in the art. Nothing herein is to be construed as an admission that the invention is not entitled to antedate such disclosure by virtue of prior invention.

The present embodiments surprisingly provide novel approaches for the conversion of biomass based char into water soluble products. The methods include subjecting biomass based char to an oxidant to provide a mixture, whereby the biomass based char of the mixture is converted into water soluble products. Generally, the biomass based char is a result of treatment of a biomass based material with a strong mineral acid, such as sulfuric, hydrochloric or an organic acid, such as methane sulfonic acid, or other catalysts and oxidants noted above, in an aqueous medium. The following list of advantages is not meant to be limiting but highlights some of the discoveries contained herein.

“Char” is thought to be a polymeric material comprising residual components of the biomass material. It is generally characterized as a dark powdery solid material to a dark sticky solid material that is found in the reaction medium as an unwanted byproduct from the production of levulinic acid from biomass materials treated with mineral acids. Up until this time, char was not studied and was discarded as an unwanted byproduct or perhaps burned for energy content. The formation of char not only reduces the yield of desired product, such as levulinic acid or formic acid, but can coat or clog the reactor with unwanted material and can also entrain desired product(s) within the char itself

Processes to Prepare Levulinic and “Char”

Conversion of biomass as an initial feedstock to prepare the levulinic acid, hydroxymethyl furfural and/or formic acid is known. For example, levulinic acid has been prepared by conversion of biomass as described in WO/2013/078391 and U.S. 61/887,657, the contents of which are incorporated herein in their entirety. This ability to utilize a wide variety of biomass provides great flexibility in obtaining a constant source of starting material and is not limiting.

Biomass comprises sludges from paper manufacturing process; agricultural residues; bagasse pith; bagasse; molasses; aqueous oak wood extracts; rice hull; oats residues; wood sugar slops; fir sawdust; naphtha; corncob furfural residue; cotton balls; raw wood flour; rice; straw; soybean skin; soybean oil residue; corn husks; cotton stems; cottonseed hulls; starch; potatoes; sweet potatoes; lactose; sunflower seed husks; sugar; corn syrup; hemp; waste paper; wastepaper fibers; sawdust; wood; residue from agriculture or forestry; organic components of municipal and industrial wastes; waste plant materials from hard wood or beech bark; fiberboard industry waste water; post-fermentation liquor; furfural still residues; and combinations thereof, furfuryl alcohol, a C5 sugar, a C6 sugar, a lignocelluloses, cellulose, starch, a polysaccharide, a disaccharide, a monosaccharide or mixtures thereof. Preferably the biomass is high fructose corn syrup, a mixture of at least two different sugars, sucrose, an aqueous mixture comprising fructose, an aqueous mixture comprising fructose and glucose, an aqueous mixture comprising hydroxymethylfurfural, an aqueous solution of fructose and hydroxymethylfurfural, an aqueous mixture of glucose, an aqueous mixture of maltose, an aqueous mixture of inulin, an aqueous mixture of polysaccharides, or mixtures thereof, and more preferably, the biomass comprises fructose, glucose or a combination thereof.

Biomass can be a refined material, such as fructose, glucose, sucrose, mixtures of those materials and the like. As such, there is a plentiful supply of materials that can be converted into the ultimate product(s). For example, sugar beets or sugar cane can be used as one source. Fructose-corn syrup is another readily available material. Use of such materials thus helps to reduce the costs to prepare desired products, such as levulinic aid, hydroxymethyl furfural and/or formic acid.

In the production of levulinic acid, use of high concentrations of acid(s), generally about 20 weight percent or more (based on the total mass of the reaction medium) provides a cleaner reaction product with less char and unwanted byproducts. It has also been found that use of high concentrations of acid(s), generally up to 75 weight percent or more, (based on the total mass of the reaction medium) provides faster reaction times than lower acid concentrations used with the same reaction conditions.

Also, in the production of levulinic acid, when higher concentrations of acid are utilized in the conversion of biomass to levulinic acid, etc., the reaction conditions can be conducted at much lower temperatures than are currently utilized in the literature. Again, this lessens the amount of char and byproducts from the reaction(s) that take place and increases the yield of the desired product(s).

The agitation in the reactors should be adequate to prevent agglomeration of solid co-products (char) which may be formed during the reaction. Specifically, the reactors should be designed with sufficient axial flow (from the center of the reactor to the outer diameter and back).

Acids

Suitable acids used to convert the biomass materials described herein, including sugars, include mineral acids, such as but not limited to sulfuric acid, hydrochloric acid, hydrobromic acid, hydroiodic acid, and organic acids, such as but not limited to methane sulfonic acid, p-toluene sulfonic acid, perchloric acid and mixtures thereof.

Flash

The reaction products of the levulinic acid process may be optionally cooled in a “flash” process. The flash step rapidly cools the reaction products by maintaining a pressure low enough to evaporate a significant fraction of the products. This pressure may be at or below atmospheric pressure. The evaporated product stream may be refluxed through stages of a distillation column to minimize the loss of desired reaction products, specifically levulinic acid, and to ensure recovery of formic acid reaction products and solvent. Recovered solvent may be recycled back to a reactor.

The “bottoms” or less volatile stream from the flash step is advanced to the solids separation stage.

Solids Separation

In the solids separation stage of the process, the solvent and desired reaction products, specifically levulinic acid and formic acid, are separated from any char which may have formed during the reaction phase. The char may be separated through a combination of centrifuge, filtration, and settling steps (ref Perrys Chemical Engineering Handbook, Solids Separation). The separated solids may be optionally washed with water and/or solvents to recover desired reaction products or solvent which may be entrained in or adsorbed to the solids. It has been found that in some embodiments, such as those reactions employing high levels of mineral acid (greater than 20%) that are reacted at lower temperatures, such as between 60-110 C, the solids may have density properties similar to the liquid hydrolysate which effectively allows the solids to be suspended in solution. In these embodiments, certain separation techniques such as centrifugation are not as effective. In these embodiments filtration utilizing filter media having a pore size less than about 20 microns has been found to effectively remove solids from the mixture. When removing solids from the system a solid “cake” is formed. It is desirable that the cake be up to 50% solids. Thus any separation technique that obtains a cake having a higher amount of solids is preferred. A certain amount of LA and mineral acid will be present in the cake and it may be desirable to wash the cake with an extraction solvent or water to recover LA.

Solid particles in the high mineral acid and lower temperature embodiments are easily filtered and do not inhibit flow as the cake is formed. It is believed that the properties of the char formed under these process conditions are such that any cake remains porous enough that a small filter size (less than 20 microns) can be utilized while maintaining a high flow rate through the medium.

Conversion of Char to Water Soluble Materials from the Levulinic Acid Processes

The present embodiments surprisingly provide novel approaches for the conversion of biomass based char into water soluble products. The methods include subjecting biomass based char to an oxidant to provide a mixture, whereby the biomass based char of the mixture is converted into water soluble products. Generally, the biomass based char is a result of treatment of a biomass based material with a strong mineral acid, such as sulfuric, hydrochloric or nitric acid in an aqueous medium as described above.

Suitable oxidants to transform the char into useful materials include, for example, a permanganate, hypochlorite, oxygen, ozone, OXONE®, nitric acid, a peroxide, as well as others described herein.

Generally the oxidant is present in an amount of from about 0.01 weight % by weight of oxidant per weight of char to about 1000 weight % by weight of char.

The reaction between the biomass based char and the oxidant is generally conducted in an aqueous environment between a temperature of from about 20° C. to about 300° C.

Generally, the weight percentage of oxidant(s) and char to water in the reaction medium varies from about 0.1 wt % to about 80 wt %.

The oxidation reaction is generally conducted in a vessel that can be stirred during the reaction. Also, the oxidation reaction can be conducted under high pressure and high temperature. Pressures can be up to 3000 psi and temperatures up to 300° C.

The oxidation reaction may be conducted in a continuous, semi-continuous, or batch-type process.

The time period for the oxidation reaction of the char is from about 1 minute to about 24 hours, more particularly from about 15 minutes to about 8 hours and even more particularly from about 30 minutes to about 6 hours. During this time, the reaction temperature can be increased over the range of temperatures noted above. Additionally, the reaction mixture can be monitored by gas chromatography, liquid chromatography, thin layer chromatography and the like. Additionally, a visual inspection of the reaction vessel will show that the solids have been solubilized such that little if any char solids remain.

During the oxidation of the char, the solid char particles are converted into water soluble compounds. Such water soluble compounds include, for example, one or more of levulinic acid, acetic acid, succinic acid, formic acid or mixtures thereof. These materials can be further isolated by methods known in the art, such as by distillation, thin film evaporation, crystallization, liquid-liquid extraction, ion exchange and adsorption. Alternatively, the water-soluble acids may be esterified with a primary alcohol, for example a C1-C18 alcohol, and purified by distillation or crystallization.

It should be understood that the oxidized product(s) can be isolated by conventional means known in the art. That is, the oxidized composition, generally in water, can be treated with a water immiscible organic solvent to remove the products from the aqueous phase. Suitable water immiscible organic solvent include, for example, polar water-insoluble solvents such as methyl-isobutyl ketone (MIBK), methyl isoamyl ketone (MIAK), cyclohexanone, o, m, and para-cresol, substituted phenols, for example, 2-sec butyl phenol, C4-C18 alcohols, such as n-pentanol, isoamyl alcohol, n-heptanol, 2-ethyl hexanol, n-octanol, 1-nonanol, cyclohexanol, methylene chloride, 1,2-dibutoxy-ethylene glycol, acetophenone, isophorone, o-methoxy-phenol, methyl-tetrahydrofuran, tri-alkylphosphine oxides (C4-C 18) and ortho-dichlorobenzene and mixtures thereof or the like, more specifically, methyl isoamyl ketone (MIAK), o, m, and para-cresol, phenol, isoamyl alcohol, n-hexanol, n-heptanol, 2-ethyl hexanol, o-methoxy-phenol, 2-4 dimethyl phenol, methyl isobutyl carbinol, and mixtures thereof or the like, and even more specifically, phenol, all isomers of fluoro, chloro, bromo, and iodo phenols, bis-halogenated phenols, mixtures of halogenated phenols, xylenols, gamma-valerolactone, isoamyl alcohol, neopentyl alcohol, methyl isobutyl carbinol, and mixtures thereof or the like Such solvents are used generally at room temperature so as not to serve as potential reaction component.

The following paragraphs, numbered 1 through 12 provide for various aspects of the present embodiments. In one embodiment, in a first paragraph (1), a method to convert biomass based char into water soluble products is provided that includes subjecting biomass based char to an oxidant to provide a mixture, whereby the biomass based char of the mixture is converted into water soluble products.

2. The method of paragraph 1, wherein the biomass based char is a result of treatment of a biomass based material with a mineral acid.

3. The method of paragraph 2, wherein the biomass based material is a C5 sugar, a C6 sugar, a lignocelluloses, cellulose, starch, a polysaccharide, a disaccharide, a monosaccharide or mixtures thereof.

4. The method of paragraph 3, wherein the sugar is a high fructose corn syrup.

5. The method of paragraph 3, wherein the sugar is glucose, fructose, sucrose or combinations thereof.

6. The method of any of paragraphs 1 through 5, wherein the oxidant is oxygen, air, a permanganate, nitric acid, or a peroxide.

7. The method of any of paragraphs 1 through 6, wherein the mixture includes water.

8. The method of any of paragraphs 1 through 7, wherein the mixture is subjected to the oxidant at a temperature of from about 20° C. to about 300° C.

9. The method of any of paragraphs 1 through 8, wherein no char remains.

10. The method of any of paragraphs 1 through 9, wherein the water soluble products are one or more of levulinic acid, acetic acid, succinic acid, formic acid or mixtures thereof.

11. The method of any of paragraphs 1 through 10, further comprising a metal containing catalyst.

12. The method of paragraph 11, wherein the catalyst comprises a metal selected from platinum, palladium, ruthenium, copper, cobalt, vanadium, tungsten, iron, silver, manganese, or gold, or a combination thereof.

The invention will be further described with reference to the following non-limiting Examples. It will be apparent to those skilled in the art that many changes can be made in the embodiments described without departing from the scope of the present invention. Thus the scope of the present invention should not be limited to the embodiments described in this application, but only by embodiments described by the language of the claims and the equivalents of those embodiments. Unless otherwise indicated, all percentages are by weight.

Analytical Methods:

HPLC. The instrument used was a WATERS 2695 LC system with a WATERS 2414 RI detector. An Aminex 87H column (300×7.8 mm) was used with 10 μL injections. An isocratic flow of 0.6 mL/min is used with a mobile phase mixture of 5 mM H₂SO₄ in Water (nanopure) and 3% Acetonitrile (HPLC grade). The column temperature was maintained at 50° C. RI detector temperature is 50° C.

Bruker Avance III 400 HD High Performance Digital NMR Spectrometer with Topspin Processing software. The NMR probe temperature was controlled at 300° K. Solvent was either CDCl₃ or D₂O.

¹H NMR 16 scans

¹³C NMR 1024 scans

LC-MS: Ascentis Express OHS 150×4.6 mm×5 micron particle diameter (HILIC mode chromatography).

Flow rate: 0.5 mL/min.

Column temp. 50° C.

Mobile phases:

Aqueous phase ˜45 mM pH 3.3 ammonium formate buffer in water.

Organic modifier was Acetonitrile (ACN)

Gradient:

| Time (min.) | % buffer | % ACN |
|---|---|---|
| 0 | 95 | 5 |
| 5 | 95 | 5 |
| 35 | 50 | 50 |
| 40 | 50 | 50 |
| 41 | 95 | 5 |
| 50 | 95 | 5 |

Mass spec conditions (negative mode operation):

ES Voltage:

| | |
|---|---|
| Capillary | 2.8 kv |
| Cone | 35 V |

Source Voltage:

| | | |
|---|---|---|
| Extractor | 3 | V |
| RF Lense | 0.2 | V |
| Source Temp. | 150° | C. |
| Desolvation Temp. | 350° | C. |
| Gas flows: | | |
| Desolvation | 800 | L/min. |
| Cone | 100 | L/min. |

GC/MS

Finnigan ThermoQuest Trace GC 2000 Series with Trace MS detector and ThermoQuest AS 2000 Autosampler. The column is Agilent Technologies HP-1 30M×0.32 mm×0.25 micrometer. Temperature ramp is to inject at 50° C., hold for 2 minutes, ramp 10° C./min to 250 and hold for 5 minutes. Trace MS detection range is set to 45 to 500 MW.

### EXAMPLES

### Examples 1-8

### Example 1

### Synthesis of Solid Bio-Char, 1

1022 lbs of deionized water and 874 lbs of 93.5% sulfuric acid were charged into a 250 gallon glass-lined reactor and heated to 105° C. 342 lbs of high fructose corn syrup (Cornsweet 90®, ADM, Inc) was added into the reactor over 2 h. The reaction mixture was heated to 120° C. for an additional 163 minutes and then cooled to 60° C. The final reaction mixture contained approximately 1.9 wt % solid bio-char (on a dry basis), as well as 4.9% levulinic acid and 2.0 wt % formic acid. After cooling, the reaction mixture was pumped out of the reactor and the solid bio-char was filtered, washed with DI water until pH>3, and dried in a Mettler Toledo HG63 Halogen Moisture Analyzer at 125° C. until the weight of the sample was constant. The solid bio-char (1) was used in subsequent experiments as detailed below.

### Example 2

### Synthesis of Wet, Acidic Bio-Char, 2

1022 lbs of deionized water and 874 lbs of 93.5% sulfuric acid was charged into a 250 gallon glass-lined reactor and heated to 105° C. 342 lbs of high fructose corn syrup (Cornsweet 90®, ADM, Inc) was added into the reactor over 2 h. A small amount of wet, acidic bio-char was isolated from the reaction system. The solid bio-char was acidic and not washed with water to lower the pH. The wet, acidic, bio-char (2) was used in subsequent experiments. The % solids content in 2 was measured to be 21.6% solids.

### Example 3

### Oxidation of 1 with H₂O₂

Into a 250 mL, 3-necked round bottom flask was added 100 mL of 3% H₂O₂ (Top Care). The flask was equipped with a magnetic stirrer, a temperature controller, and a reflux condenser. 1.4 grams of solid bio-char (1) was added into the reactor, and the reactor was heated to 40° C. Then, 4.2 grams of sodium tungstate dehydrate was added into the reactor. The mixture was black and had solid char dispersed in the mixture. After the addition of the sodium tungstate, bubbling and foam developed due to the generation of oxygen. The reaction exothermed to 52-54° C. After, 1 hour (h), the reaction was heated to 65° C. The solution became more reddish in color. The reaction mixture was then heated to 85° C., and after 1 h at 85° C., the solution became red and transparent. The black bio-char solids had oxidized into water-soluble compounds. The temperature was increased to 100° C., and heated for 4 h at 90-100° C. The solution became darker red over the course of the 4 h reflux, yet the solution remained transparent and free of solids. The reaction had converted 100% of the solids into oxidized or soluble products. Aliquots were taken from the reactor, the peroxides were quenched with sodium thiosulfate, and the sample was diluted in the HPLC mobile phase (mobile phase: 20 mM Phosphoric acid in deionized water with 3% Acetonitrile). LC analysis showed 3 new peaks in the aliphatic acid region of the chromatograph. One of the unknown peaks had a retention time similar to succinic acid. There was also a peak with a retention time similar to acetic acid and levulinic acid. A portion of the reaction mixture was acidified to pH 3-4, and the water was distilled off under vacuum. A solid precipitate formed. The precipitate was analyzed by LC and it was found to contain a broad peak in the organic acid region in the LC, as well as, inorganic salt impurities from the tungstate and thiosulfate. Upon dilution of the sample, the broad peak in the organic acid region separated into the organic acid peaks described above (succinic, acetic/levulinic, and unknown peaks).

### Example 4

### Oxidation of 2 with H₂O₂

Into a 250 mL, 3-necked round bottom flask was added 70 g of 3% H₂O₂ (Top Care). The flask was equipped with a magnetic stirrer, a temperature controller, and a reflux condenser. 6.7 grams of 2 was added into the reactor, and the reactor was kept at 20° C. Then, 0.8 grams of sodium tungstate dehydrate was added into the reactor. The mixture was black and had solid char dispersed in the mixture. The reaction was heated from 20° C. to 85° C. over 2.5 h. The reaction mixture was still mostly black, but it was became dark reddish in color. The temperature was increased to 100° C., and heated for 2 h. The solution became redder in color, but it remained translucent in nature instead of completely transparent red. There were filterable solids in the reaction mixture. LC analysis showed the presence of levulinic acid, formic acid, glucose, fructose, and 3 new peaks (10.7 min, 11.5 min, and 12.3 min) that were different from the initial chromatograph. One of the new peaks displayed a retention time that was similar to the retention time of succinic acid (11.5 min). It should be noted that the levulinic acid peak (retention time=15.2 min) increased in area, and this may be due to increasing amounts of levulinic acid or the formation of acetic acid which has a similar retention time on the LC. The reaction was halted, cooled and filtered. The weight percent conversion of solid reactants (2) into oxidized or soluble products was measured to be 55.9% (on a dry solids basis).

### Example 5

### Oxidation of 1 with HNO₃

Into a 250 mL, 3-necked round bottom flask was added 50 g of DI H₂O and 64 g of 65-70% HNO₃. The flask was equipped with a magnetic stirrer, a temperature controller, and a reflux condenser. 4.8 grams of 1 was added into the reactor, and the reactor was kept at 20° C. A small exotherm was noticed from 29-35° C. Then, 3 g of Cu₂O was added and the mixture exothermed to 44° C. Contents bubbled, and the appearance of NO₂ was noticed (brown gas). The flask was heated to 60° C. for 30 min, and then heated to 70° C. for 2 h. The mixture changed color from dark black with dispersed solids to light brown and transparent. LC analysis of the reaction mixture showed the presence of 2 new peaks (12 min and 12.7 min), plus a peak with a retention time similar to succinic acid (11.5 min). Peaks that corresponded to levulinic acid or acetic acid (retention time=15.2 min) and formic acid (retention time=14.1 min) were also noticed. An LC-MS was performed, and it did not show the presence of levulinic acid, so the peak at 15.2 min, was acetic acid. After the reaction the Cu₂O catalyst was filtered away, a dark transparent solution was observed. The weight percent conversion of 1 to oxidized or soluble products was 100%.

### Example 6

### Oxidation of 1 with H₂O₂

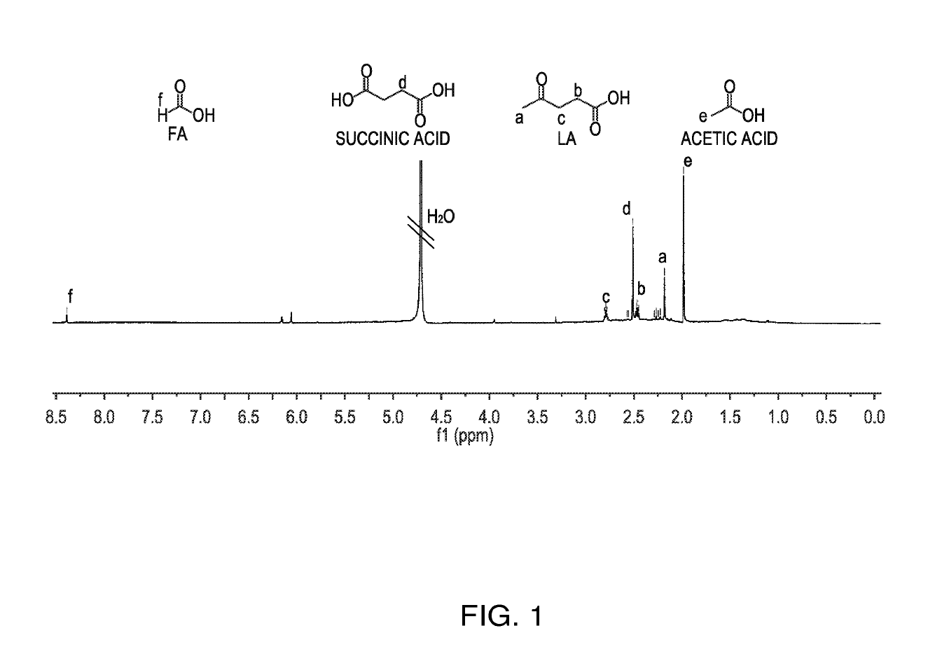

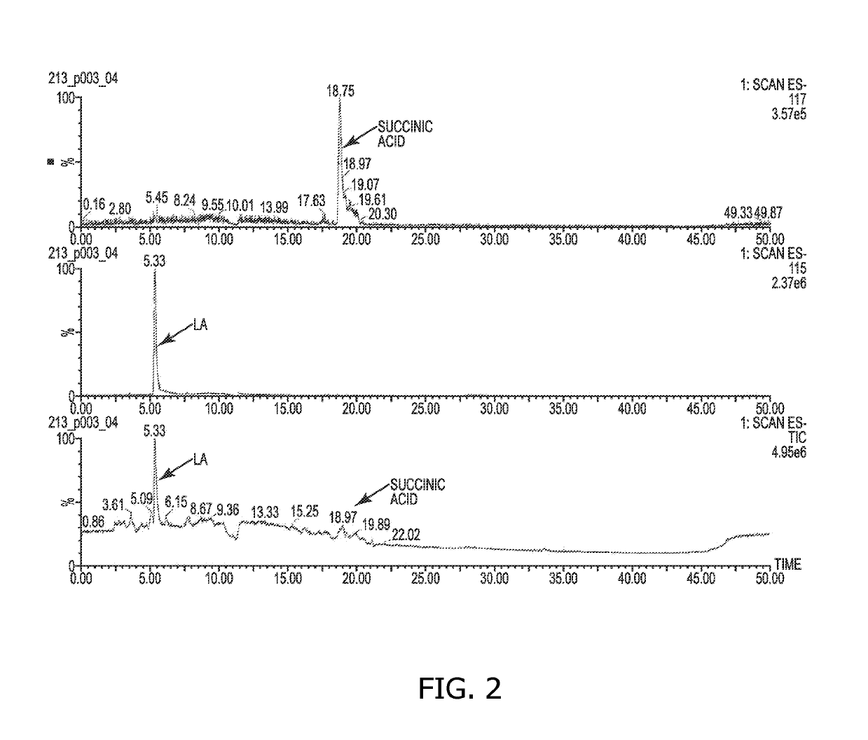

Into a 250 mL, 3-necked round bottom flask was added 100 g of 3% H₂O₂ (Top Care). The flask was equipped with a magnetic stirrer, a temperature controller, and a reflux condenser. 1 gram of solid bio-char (1) was added into the reactor, and the reactor was put into an ice bath. Then, 0.85 grams of sodium tungstate dehydrate was added into the reactor. The mixture was black and had solid char dispersed in the mixture. The ice bath was removed and the reaction was heated to 85° C. over 40 min. The reaction mixture was heated at 85° C. for 60 min, and the solution turned from black to reddish-brown, but it was not transparent. The reaction was tested for peroxides and the presence of peroxides was confirmed. The flask was cooled and 0.8 g of sodium tungstate dehydrate was added to the flask, the flask was then heated to 65° C. and held for 90 min. The reaction mixture turned brown and had dispersed solids. The reaction mixture was heated to 85° C., and after 2 h the solution was dark orange and transparent. After 4 h at 85° C., the reaction mixture was lighter in color (bright orange), and transparent. Analysis of the reaction mixture by LC confirmed the presence of acidic products similar to Example 3. (See FIG. 2) The black bio-char solids had oxidized into water-soluble compounds. The reaction had converted 100% of the solids into oxidized or soluble products. The water was removed overhead by vacuum distillation on a rotary evaporator at 85° C. The overhead sample contained acetic acid, measured by LC. An NMR of the concentrated bottoms sample was analyzed by proton and carbon NMR. The NMR spectra confirmed the presence of levulinic acid, formic acid, acetic acid, succinic acid. (See FIG. 1.) There were unknown compounds in the NMR analysis also. The NMR analysis confirmed the HPLC analysis that there was succinic acid, levulinic acid, formic acid, and acetic acid, as well as, unknown compounds were made from the peroxide oxidation of 1.

### Example 7

### Oxidation of 1 with KMnO₄

Into a 250 mL, 3-necked round bottom flask was added 91.6 g of DI H₂O, 6.0 g of KMnO₄, and 1.0 g of 1. 5 g of DI water was used to rinse the funnel. The flask was equipped with a magnetic stirrer, a temperature controller, and a reflux condenser. The mixture was black (dark purple). The heating mantle was turned on to heat the flack and the mixture exothermed to 67° C. The flask was heated to 85° C. for 4 h and sampled. The LC showed levulinic acid/acetic acid, formic acid, and succinic acid. However, the mixture was not clear and transparent, it was orange and transparent. The pH of the reaction mixture was adjusted to 6 with H₂SO₄ and heated back up to 85° C. The reaction mixture did not filter into a clear and transparent solution. An additional 9 g of KMnO₄ was added to the flask, and the contents were heated to 85-90° C. for 6 h. 2 g of methanol was added to decompose the excess KMnO₄ into MnO₂, and the reaction was filtered into a water-white, transparent solution. LC showed the presence formic acid, acetic/levulinic acid, and perhaps a small amount of succinic acid. The volatiles were removed by rotary evaporation under vacuum, and a white crystalline solid developed. A sample of the material was dissolved in D₂O and analyzed by proton and carbon NMR. NMR showed formic acid and acetic acid, and no other compounds as detected by NMR.

### Example 8

A mixture of 5 mL water, 0.75 grams KMnO₄, and 0.1 grams 1 was placed in a 25 ml round bottom flask equipped with magnetic stirring bar and air condenser. This was placed in an oil bath, stirred, and heated to 85° C. A vigorous reaction occurred as the mixture reached 80° C. The purple liquid became black, and then brown/red. After 3 hours, a 1 mL aliquot of methanol was added to the reaction mixture to reduce remaining unreacted permanganate. The flask was removed from the oil bath and allowed to cool to room temperature. The black mixture was filtered to remove solids, and the resulting nearly colorless solution was taken to dryness on a rotary evaporator. The solids were analyzed by ¹H and ¹³C NMR and by GC-MS. Both methods, NMR and GC-MS, show the presence of levulinic acid, succinic acid, acetic acid, and formic acid.

### GC-FID Method

The GC-FID analysis was performed on a Restek Stabilwax-DA (15 m×0.25 mm(ID)×0.25 μm) column using Helium as a carrier gas with a flow rate of 1 mL/min. The initial oven temp was held at 160° C. for 1.5 min. The oven was then ramped at 10° C./min. to 200° C., and then ramped at 20° C./min. to a final temp of 250° C. and held at 250° C. for 10 min. The injection port temperature was 250° C. and the split ratio was 50:1. The FID detector was set at 250° C. with the following flow rates: hydrogen at 30 mL/min, air at 400 mL/min, and helium make-up at 25 mL/min. An injection volume of 1 μL was used for all samples. The identification and quantification of acetic acid, propanoic acid, and levulinic acid was performed using standards prepared in THF.

### SEC Method for Tars

The size-exclusion chromatography (SEC) analysis was performed on an HPLC with refractive index (RI) detection. Three columns were used in series (columns 1 and 2: Agilent PLGel, 3 μm 100A, 300×7.5 mm; column 3: Tosoh TSKGel G1000HHR, 5 μm, 300×7.8 mm). The columns were maintained at 35° C., and the flow rate was 1 mL/min. The mobile phase was unstabilized THF. The RI detector was maintained at 40° C. Injection volume of 10 μL was used for all samples. The final SEC trace of the reaction mixture was integrated from 14.5 min. to 22.5 min. to determine the concentration of soluble oligomers (tars) in the final reaction media using a purified tar standard.

### HPLC Method for Acids

An HPLC method was used for the analysis of a variety of organic acids. The method used a reverse-phase column with UV-Vis detection. The column used was a Restek Ultra C18-Aq (150×3.2 mm, 3 μm), and maintained at 30° C. The initial mobile phase conditions were 50 mM Potassium Phosphate Monobasic, with 1% Acetonitrile. Then initial conditions were held for 5 min., and a gradient was performed to 60% acetonitrile from 5 to 13 min. The flow rate was 0.7 mL/min. A photodiode array detector was used at a wavelength of 210 nm. Weak acid peaks were identified and quantified using a mixture of acid standards in water. Samples were prepared from reaction mixtures in water and filtered before injection. Injection volume was 10 μL. Examples 9-21: Oxidation of tar using H₂O₂

### Examples 9-21

To a capillary tube sealed at one end was added a solution containing 5% Tar (w/v), 5% H₂O₂ (w/v), 45% H₂O (v/v) and 45% Acetic Acid (w/v), and between 0.01 and 0.1 M catalyst, as described in the table below. The solutions were added to the capillaries till the tubes were approximately ⅔ full. The tubes were then placed in an Al heating block and flame sealed using a hand-held butane torch. The samples were then transferred to a pre-heated Al heating block. The sealed capillaries were reacted at the desired temperature for 2 hours before being removed to cool. The capillaries were un-sealed and the resulting reaction mixtures were sampled for analysis. The summary of those analyses is presented in the table below:

| | | Concentration | | | | % Tar |
|---|---|---|---|---|---|---|
| Example | Catalyst | (M) | Temp (° C.) | % LA | % SA | Conversion |
| 9 | NiSO₄•H₂O | 0.1 | 100 | 0.60 | 0.06 | 69.0 |
| 10 | FeSO₄•7H₂O | 0.01 | 100 | 0.67 | 0.09 | 68.6 |
| 11 | CuSO₄•5H₂O | 0.1 | 100 | 0.48 | 0.11 | 74.0 |
| 12 | Fe₂(SO₄)₃•H₂O | 0.01 | 100 | 0.58 | 0.05 | 75.2 |
| 13 | FeCl₃ | 0.01 | 100 | 0.61 | 0.07 | 72.2 |
| 14* | p-TsOH | 0.1 | 80 | 0.71 | 0.05 | 53.4 |
| 15 | Na₂WO₄•2H₂O | 0.1 | 100 | 0.68 | 0.06 | 66.4 |
| 16 | H₃PW₁₂O₄₀•H₂O | 0.01 | 100 | 0.74 | 0.07 | 61.2 |
| 17 | Cr₂(SO₄)₃•H₂O | 0.01 | 100 | 0.66 | 0.12 | 75.4 |
| 18 | MnSO₄•H₂O | 0.1 | 100 | 0.68 | 0.02 | 65.0 |
| 19 | CoSO₄•H₂O | 0.1 | 100 | 0.30 | 0.09 | 74.2 |
| 20 | VOSO₄•H₂O | 0.1 | 100 | 0.59 | 0.05 | 72.2 |
| 21 | None | | 100 | 0.58 | 0.03 | 70.2 |

*The capillary reactor containing p-TsOH ruptured during the 100° C. heating phase. For this reason, a lower temperature example is shown.
(LA = levulinic acid; SA = succinic acid)

### Example 22

### Large Scale Char Oxidation

To a 50 mL round bottom flask was charged 2.044 g Char, 17.033 g acetic acid, 6.807 g 30% H₂O₂, and 13.638 g H₂O. The flask was heated with agitation in a 100° C. oil bath for 4 hours. After 4 hours, the flask was removed from the oil bath and cooled to room temperature. The reaction mixture was then sampled for analysis. The results of that analysis are shown below:

| | SEC Analysis | LC Analysis |
|---|---|---|
| Component | (%) | (%) |
| Tar | 55.9 | — |
| Acetic Acid | — | 10.6 |
| Propanoic Acid | — | 0.1 |
| Levulinic Acid | — | 11.9 |
| Succinic Acid | — | 2.5 |
| Maleic Acid | — | 0.03 |
| Lactic Acid | — | 0.2 |
| Itaconic Acid | — | 0.1 |
| Butyric Acid | — | 10.6 |
| Oxalic Acid | — | 0.1 |
| SUM | — | 92.3 |

### Example 23-26

### High Pressure/Temperature Tar Oxidation

In a typical high temperature tar oxidation, a 6 mL Hastelloy C-276 tube reactor was charged with 3 mL of a reaction mixture comprising 5% (w/v) tar, 5% or 15% (w/v) H₂O₂, 45% (w/v) acetic acid, with the remain consisting of water. The reactor tubes were sealed and placed in a sand bath within an oven for 4 hours. After 4 hours, the heat to the oven was shut off and the reactors were allowed to cool to room temperature inside the oven. Once cool, the samples were removed from the oven and carefully opened behind a small blast shield. The reaction mixture within was samples and analyzed, with the results of those analyses shown below:

| | | | | % Tar | % LA |
|---|---|---|---|---|---|
| | | | Temp | Conversion | Selectivity |
| Example | % Tar | % H₂O₂ | (° C.) | (SEC) | (GC) |
| 23 | 5 | 5 | 140 | 69.0 | 18.9 |
| 24 | 5 | 15 | 140 | 90.3 | 11.8 |
| 25 | 5 | 5 | 180 | 59.3 | 25.4 |
| 26 | 5 | 15 | 180 | 76.0 | 14.2 |

Although the present invention has been described with reference to preferred embodiments, persons skilled in the art will recognize that changes may be made in form and detail without departing from the spirit and scope of the invention. All references cited throughout the specification, including those in the background, are incorporated herein in their entirety. Those skilled in the art will recognize, or be able to ascertain, using no more than routine experimentation, many equivalents to specific embodiments of the invention described specifically herein. Such equivalents are intended to be encompassed in the scope of the following claims.

What is claimed is:

1. A method to convert biomass based char into water soluble products comprising the step:
subjecting biomass based char to an oxidant to provide a mixture, whereby the biomass based char of the mixture is converted into water soluble products.

2. The method of claim 1, wherein the biomass based char is a result of treatment of a biomass based material with a mineral acid.

3. The method of claim 2, wherein the biomass based material is a C5 sugar, a C6 sugar, a lignocelluloses, cellulose, starch, a polysaccharide, a disaccharide, a monosaccharide or mixtures thereof.

4. The method of claim 3, wherein the sugar is a high fructose corn syrup.

5. The method of claim 3, wherein the sugar is glucose, fructose, sucrose or combinations thereof.

6. The method of claim 1, wherein the oxidant is oxygen, air, a permanganate, nitric acid, or a peroxide.

7. The method of claim 1, wherein the mixture includes water.

8. The method of claim 7, wherein the mixture is subjected to the oxidant at a temperature of from about 20° C. to about 300° C.

9. The method of claim 1, wherein no char remains.

10. The method of claim 1, wherein the water soluble products are one or more of levulinic acid, acetic acid, succinic acid, formic acid or mixtures thereof.

11. The method of claim 1, further comprising a metal containing catalyst.

12. The method of claim 11, wherein the catalyst comprises a metal selected from platinum, palladium, ruthenium, copper, cobalt, nickel, vanadium, tungsten, iron, silver, manganese, or gold, or a combination thereof.
